(19) United States
(12) Patent Application Publication
(10) Pub. No.: US 2014/0088332 A1
(43) Pub. Date: Mar. 27, 2014

(54) PROCESS FOR THE PRODUCTION OF 1,3-BUTADIENE IMPLEMENTING THE OLIGOMERIZATION OF ETHYLENE AND THE DEHYDROGENATION OF THE BUTENES THAT ARE OBTAINED

(71) Applicant: AXENS, Rueil Malmaison (FR)

(72) Inventor: Gildas Rolland, Rueil Malmaison (FR)

(73) Assignee: AXENS, Rueil Malmaison (FR)

(21) Appl. No.: 14/033,953

(22) Filed: Sep. 23, 2013

(30) Foreign Application Priority Data
Sep. 21, 2012 (FR) 12/02.510

(51) Int. Cl. C07C 5/48; C07C 2/06

(52) U.S. Cl. CPC C07C 5/48; C07C 2/06
USPC 585/326

(57) ABSTRACT

This invention describes a process for the production of 1,3-butadiene from ethylene implementing a stage for oligomerization of ethylene into n-butenes and into oligomers with 6 carbon atoms and more by homogeneous catalysis, a stage for separation in such a way as to obtain an n-butene-enriched fraction, and then a stage for dehydrogenation of said n-butene-enriched fraction.

This invention describes a process for the production of 1,3-butadiene from ethylene implementing a stage for oligomerization of ethylene into n-butenes and into oligomers with 6 carbon atoms and more by homogeneous catalysis, a stage for separation in such a way as to obtain an n-butene-enriched fraction, and then a stage for dehydrogenation of said n-butene-enriched fraction.

### PRIOR ART ON THE PRODUCTION OF BUTADIENE

Currently, 1,3-butadiene is for the most part produced by extraction of the C4 fraction resulting from the steam-cracking of naphtha, in general by extractive distillation using acetonitrile or N-methyl pyrrolidone, or by liquid-liquid extraction.

It is also prepared by catalytic dehydrogenation of butenes and butane (Houdry process, now CATADIENE™ of the Lummus Company).

In addition to the dehydrogenation of C4 hydrocarbons into butadiene, another dehydrogenation method (in the presence of oxygen) has gained importance.

Thus, the Phillips O-X-D™ process (oxidizing dehydrogenation) for the manufacturing of butadiene from n-butenes is an example of an industrially exploited dehydrogenation process. The n-butenes, from water vapor and air, are caused to react at 480-600° C. in a fixed-bed catalyst. This process makes it possible to attain conversions of butenes between 75 and 80% and attains a butadiene selectivity of approximately 88-92%. This process was used by Philips until 1976.

The Petro-Tex Company also developed a process for the oxidizing dehydrogenation of butenes (Oxo-D™ process). The conversion of oxygen or air is carried out at 550-600° C. on a heterogeneous catalyst (ferrite with Zn, Mn or Mg). By adding vapor for controlling the selectivity, a butadiene selectivity of 93% (based on n-butenes) can be obtained with a conversion of 65%.

The most important source of butadiene is the C4 fraction resulting from the steam-cracking of naphtha, with butadiene being produced as a by-product in the production of ethylene. The increase in the ethylene production for the most part is supported by the capacities of the steam-cracking devices of which it is noted that the feedstock shifts toward a larger contribution of ethane, for the sake of the economic advantage of ethane, available in a large quantity and at a lower price relative to other feedstocks. Taking into account the butadiene to ethylene production ratio that decreases when the steam-cracking device is supplied by feedstocks containing more ethane, the availability of butadiene therefore increases less quickly, which results in tension on the butadiene market and the necessity for diversifying the supply sources.

Thus, alternative processes for the production of butadiene have been studied. The document JP2011/148720 describes a process for the production of butadiene comprising a stage for dimerization of ethylene into butenes on a heterogeneous catalyst, followed by oxidizing dehydrogenation. The first stage, the dimerization of ethylene, is carried out in the presence of a heterogeneous catalyst that consists of Ni, alumina, and silica at a temperature of between 150 and 400° C. The second stage, the oxidizing dehydrogenation of the butenes that are obtained, is performed between 300 and 600° C. in the presence of oxygen and a complex metal oxide comprising molybdenum and bismuth. The innovation of this process scheme resides in the discovery of a heterogeneous catalyst of more stable dimerization having a low nickel content (less than 1% by weight) and a certain Ni/Al and Si/Al ratio.

It is indicated in this application that the use of the specific catalyst described in JP2011/148720 makes it possible to minimize the problems of heterogeneous catalysts that are generally known for this reaction, in particular for lowering the quantity of isobutene produced during the dimerization. Actually, the butene that is obtained by dimerization of ethylene with a heterogeneous process usually contains quantities of excess isobutene so that separation of isobutene by distillation is possible. The production of isobutene is an undesirable reaction because isobutene is difficult to separate from butenes that are produced. In addition, it is transformed into acrolein during the oxidizing dehydrogenation reaction, thus requiring complex stages for separation and purification of butadiene. The use of the specific catalyst also makes it possible, according to JP2011/148720, to minimize the deactivation of the dimerization catalyst by coking, because the stage for dimerization by heterogeneous catalysis is to be performed, according to JP2011/148720, at temperatures above 150° C.

Although the catalyst according to JP2011/148720 made it possible to minimize the problems encountered in heterogeneous catalysis, it does not make it possible to eliminate them completely.

The purpose of this invention is to improve the process for production of butadiene via a stage for oligomerization of ethylene, followed by a stage for separation of the C4 fraction containing the n-butenes, and a dehydrogenation stage by using a homogeneous catalyst in the first stage.

Actually, the use of a homogeneous catalyst during the first oligomerization stage offers the advantage of obtaining a high yield relative to the overall ethylene, on the one hand, a very good yield toward the n-butenes (typically between 30 and 60%) with an almost-zero isobutene formation, and, on the other hand, a formation of oligomers with 6 carbon atoms and more that can be upgraded in a different way than for the production of 1,3-butadiene, for example as comonomers for polymerizations or as compounds that can be introduced into a fuel pool. This minimizes the costs associated with the recycling of unconverted ethylene that characterizes other processes. The process according to the invention is therefore distinguished from the low conversions per pass and high recycling stages that are necessary in the heterogeneous processes.

Likewise, the use of a homogeneous catalyst in the oligomerization stage makes it possible to work at low temperatures (typically below 180° C.). This offers, apart from the obvious advantage of reducing the heat supply (and therefore costs), the following advantages:
The product of the first stage is obtained in liquid form that can be stored directly: this makes it possible to increase the operability and the flexibility of the overall process because it is possible, if there is concern in the second stage, to easily store the product of the first stage,
Homogeneous oligomerization does not require a means for eliminating coke by burning because there is no heterogeneous catalytic bed involved,
When the variant of the process according to the invention is such that the n-butene-enriched fraction primarily contains butene-1, a portion of this fraction can itself be upgraded as such for other applications than those described here (for example, as raw material for copolymerizations of linear low-density polyethylene type (PEBDL or LLDPE of “linear low-density polyethylene” in English) or of high-density polyethylene type (PEHD, or HDPE of “high-density polyethylene” in English)), taking into account its very high content of butene-1.

In addition, one of the most striking advantages of a first stage by homogeneous catalysis is the selectivity of the process. The formation in the C4 isobutene fraction or other branched compounds that are unfavorable in the second stage is virtually non-existent in the stage for oligomerization by homogeneous catalysis. The process is extremely selective toward the linear olefins. In particular, the isobutene does not have to be eliminated since the n-butenes that are obtained from the oligomerization do not contain it.

Below, n-butenes are defined as butene-1, cis-butene-2, and trans-butene-2.

### DESCRIPTION OF THE PROCESS

This invention describes a process for the production of 1,3-butadiene from a stream that comprises ethylene implementing the following stages:
a) An oligomerization of ethylene into oligomers is carried out by bringing said stream into contact with a catalytic system based on a homogeneous catalyst, in such a way as to produce an effluent that comprises n-butenes and oligomers with 6 carbon atoms and more including n-hexenes, n-octenes, and n-decenes,
b) A separation of the effluent obtained in stage a) is carried out in such a way as to obtain an n-butene-enriched fraction,
c) Dehydrogenation of said n-butene-enriched fraction obtained in stage b) is carried out by bringing at least a portion of said effluent into contact with a heterogeneous catalyst, in such a way as to produce an effluent comprising 1,3-butadiene.

a/ Oligomerization of Ethylene into n-Butenes and into Oligomers with 6 Carbon Atoms and More

The first stage of the process according to the invention comprises the oligomerization of ethylene into C4 dimers and into oligomers with 6 carbon atoms and more in the presence of a homogeneous catalyst in such a way as to produce an effluent that comprises n-butenes, and oligomers with 6 carbon atoms and more.

The oligomerization of ethylene into C4 dimers and into oligomers with 6 carbon atoms and more can be carried out by any homogeneous catalytic process known from the prior art, and among the latter, those that lead to a high selectivity of linear olefins are preferably selected.

a1/ By a Nickel-Based Homogeneous Catalytic System

According to a first variant, the oligomerization of ethylene is implemented in the presence of a catalytic system in the liquid phase comprising a nickel compound and an aluminum compound. Such catalytic systems are described in the documents FR 2 443 877 and FR 2794 038. The Dimersol E™ process, marketed by the Axens Company, is based on this technology and leads to the industrial production of olefins.

Thus, according to this variant, the oligomerization of ethylene is implemented in the presence of a catalytic system comprising:
i) At least one bivalent nickel compound,
ii) At least one hydrocarbyl aluminum dihalide of formula AlRX₂, in which R is a hydrocarbyl radical comprising 1 to 12 carbon atoms, such as alkyl, aryl, aralkyl, alkaryl or cycloalkyl, X is a chlorine or bromine atom, and
iii) Optionally a Brønsted organic acid.

As a bivalent nickel compound, it is possible to use all of the soluble compounds with more than 1 g per liter of hydrocarbon-containing medium, and more particularly in the reagents and the reaction medium. Preferably, the nickel carboxylates of general formula (R₁COO)₂Ni are used, where R₁ is a hydrocarbyl radical, for example alkyl, cycloalkyl, alkenyl, aryl, aralkyl, or alkaryl, containing up to 20 carbon atoms, preferably a hydrocarbyl radical of 5 to 20 carbon atoms. The radical R₁ can be substituted by one or more halogen atoms; hydroxy, ketone, nitro, or cyano groups; or other groups that do not interfere with the reaction. The two radicals R₁ also can constitute an alkylene radical of 6 to 18 carbon atoms. Nonlimiting examples of nickel compounds are the following bivalent nickel salts: chloride, bromide, carboxylates such as octoate, 2-ethylhexanoate, decanoate, oleate, salicylate, hydroxydecanoate, stearate, phenates, naphthenates, and acetyl acetonates. Nickel 2-ethylhexanoate is preferably used.

The hydrocarbyl aluminum dihalide compound corresponds to the formula AlRX₂, in which R is a hydrocarbyl radical comprising 1 to 12 carbon atoms, such as alkyl, aryl, aralkyl, alkaryl or cycloalkyl, and X is a chlorine or bromine atom. As examples of such compounds, it is possible to mention ethylaluminum sesquichloride, dichloroethyl aluminum, dichloroisobutyl aluminum, chlorodiethyl aluminum or mixtures thereof.

According to a preferred method, a Brønsted organic acid is used. The Brønsted acid compound corresponds to the formula HY, where Y is an organic anion, for example carboxylic, sulfonic or phenolic. The acids are preferred whose pKₐ at 20° C. is at a maximum equal to 3, more particularly those that are more soluble in the nickel compound or in its solution in a hydrocarbon or another suitable solvent. A preferred acid class comprises the group that is formed by the halocarboxylic acids of formula R₂COOH in which R₂ is a halogenated alkyl radical, in particular those that contain at least one alpha-halogen atom of the group —COOH with 2 to 10 carbon atoms in all. Preferably, a haloacetic acid of formula CXₚH₃₋ₚ—COOH is used, in which X is fluorine, chlorine, bromine or iodine, with p being an integer from 1 to 3. By way of example, it is possible to cite the trifluoroacetic, difluoroacetic, fluoroacetic, trichloroacetic, dichloroacetic, and chloroacetic acids. These examples are not limiting, and it is also possible to use arylsulfonic, alkylsulfonic, and fluoroalkylsulfonic acids, and picric acid and nitroacetic acid. Trifluoroacetic acid is preferably used.

The three components of the catalytic formula can be mixed in any order. However, it is preferable first to mix the nickel compound with the Brønsted organic acid, and then next to introduce the aluminum compound. The molar ratio of the hydrocarbyl aluminum dihalide to the nickel compound, expressed by the Al/Ni ratio, is 2/1 to 50/1, and preferably 2/1 to 20/1. The molar ratio of the Brønsted acid to the nickel compound is 0.25/1 to 10/1, and preferably 0.25/1 to 5/1.

According to a preferred method, the hydrocarbyl aluminum dihalide can be enriched with an aluminum trihalide, the mixture of the two compounds then corresponding to the formula AlR-ₙX₃₋ₙ, in which R is a hydrocarbyl radical comprising 1 to 12 carbon atoms, such as alkyl, aryl, aralkyl, alkaryl or cycloalkyl, X is a chlorine or bromine atom, and n is a number between 0 and 1.

The mixtures are obtained by mixing a hydrocarbyl aluminum dihalide of formula AlRX₂, in which R is a hydrocarbyl radical comprising 1 to 12 carbon atoms, such as alkyl, aryl, aralkyl, alkaryl or cycloalkyl, and X is a chlorine or bromine atom, with an aluminum trihalide AlX₃, whereby X is a chlorine or bromine atom. Without the following list being limiting, it is possible to cite such compounds by way of example: dichloroethyl aluminum enriched with aluminum chloride, the mixture having a formula AlEt_{0.9}Cl_{2.1}; the dichloroisobutyl aluminum enriched with aluminum chloride, the mixture having a formula AliBu_{0.9}Cl_{2.1}; and the dibromoethyl aluminum enriched with aluminum bromide, the mixture having a formula AlEt_{0.9}Br_{2.1}.

Also in this case, the three components of the catalytic formula can be mixed in any order. It is also preferable first to mix the nickel compound with the Brønsted organic acid, and then next to introduce the aluminum compound. In this case, this is the molar ratio between the hydrocarbyl aluminum dihalide that is enriched with aluminum trihalide, and the nickel compound, expressed by the Al/Ni ratio, which is 2/1 to 50/1, and preferably 2/1 to 20/1.

The molar ratio of the Brønsted acid to the nickel compound is also, as indicated above, from 0.25/1 to 10/1, and preferably from 0.25/1 to 5/1.

According to a preferred method, the components that are described above are mixed, in a solvent, at a controlled temperature and for a specified period of time, which constitutes a so-called pre-conditioning stage before their use in the reaction as described in the patent FR2794038. More specifically, this pre-conditioning of the catalytic composition consists in carrying out the mixing of the three components in a hydrocarbon-containing solvent, for example an alkane or an aromatic hydrocarbon, or else a halogenated hydrocarbon, or else in a preferred way the olefins that are produced in the oligomerization reaction, while being stirred and under an inert atmosphere, for example under nitrogen or under argon, at a controlled temperature of between 0 and 80° C., preferably between 10 and 60° C., for a period of 1 minute to 5 hours, preferably 5 minutes to 1 hour. The thus obtained solution is then transferred under an inert atmosphere into the oligomerization reactor.

According to this first variant, the reaction for oligomerization of ethylene can be implemented at a temperature of −20 to 80° C., preferably 40 and 60° C., under pressure conditions such that the reagents are kept at least for the most part in the liquid phase or in the condensed phase. The pressure is generally between 0.5 and 5 MPa, preferably between 0.5 MPa and 3.5 MPa. The time of contact is generally between 0.5 and 20 hours, preferably between 1 and 15 hours.

The oligomerization stage can be implemented in a reactor with one or more reaction stages in a series, with the ethylene feedstock and/or the catalytic composition that is preferably pre-conditioned in advance being introduced continuously, either in the first stage, or in the first stage and any other one of the stages. At the outlet of the reactor, the catalyst can be deactivated, for example by injection of ammonia and/or an aqueous solution of soda and/or an aqueous solution of sulfuric acid. The unconverted olefins and alkanes that are optionally present in the feedstock are then separated from the oligomers by a separation stage, for example by distillation or washing cycles by means of caustic soda and/or water.

In the case of this first variant, the oligomerization of ethylene is implemented in the presence of a catalytic system comprising a catalyst that is based on nickel and an aluminum compound. The conversion per pass is then generally 85 to 98%. The selectivity of n-butenes that are formed is generally between 50 and 80%. The n-butenes consist of butene-2 (cis- and trans-) and butene-1.

The effluent generally contains less than 0.2% by weight of isobutene, and even less than 0.1% by weight of isobutene.

The oligomerization reaction can be carried out in the presence of an ionic liquid as described in the French patent FR-B-2 611 700. The liquids with an ionic nature formed by aluminum halides of the AlRₙX₃₋ₙ formula type—in which R is a hydrocarbyl radical comprising 1 to 12 carbon atoms, such as alkyl, aryl, aralkyl, alkaryl or cycloalkyl, X is a chlorine or bromine atom, and n is a number between 0 and 1—and quaternary ammonium halides are used as solvents of nickel complexes. The use of these ionic media that are not miscible with the aliphatic hydrocarbons, in particular with the products obtained from the dimerization of olefins, thus makes possible a better use of the homogeneous catalysts.

a2/ By a Zirconium-Based Homogeneous Catalytic System

According to a second variant, the oligomerization of ethylenes is implemented in the presence of a catalytic system comprising a zirconium compound and an aluminum compound. Such catalytic systems are described in the document EP 0 578 541. The AlphaSelect™ process, marketed by the Axens Company, is based on this technology and leads to the industrial production of linear oligomers and in particular linear alpha-olefins.

Thus, according to this variant, the oligomerization of ethylene is implemented in the presence of a catalytic system comprising:
i) At least one zirconium compound of formula ZrXₓY_{y}O_{z} in which X is a chlorine or bromine atom, Y is a radical that is selected from the group that is formed by the RO— alkoxys, the R₂N— amidos, and the RCOO— carboxylates, where R is a hydrocarbyl radical comprising 1 to 30 carbon atoms, and x and y can assume the integer values of 0 to 4, and z is equal to 0 or 0.5, with the sum x+y+2z being equal to 4,
ii) At least one organic compound of formula

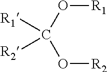

in which R′₁ and R′₂ consist of a hydrogen atom or a hydrocarbyl radical comprising 1 to 30 carbon atoms; R₁ and R₂ are hydrocarbyl radicals comprising 1 to 30 carbon atoms,
iii) And at least one aluminum compound of formula AlR″ₙX₃₋ₙ, in which R″ is a hydrocarbyl radical comprising 1 to 6 carbon atoms, X is a chlorine or bromine atom, and n is a number between 1 and 2.

The catalysts that are obtained by mixing a zirconium compound with at least one organic compound selected from among the class of aldehyde acetals and ketone cetals, and with at least one particular aluminum compound, have an unexpected selectivity for the formation of lower oligomers, primarily butene-1, hexene-1, octene-1 and decene-1. In addition to the improvement of the selectivity for the light alpha-olefins, the catalysts also have the object of reducing the by-product polymer to a very small amount.

The zirconium compounds used in the invention correspond to the general formula ZrXₓY_{y}O_{z}, in which X is a chlorine or bromine atom, and Y is a radical that is selected from among the RO— alkoxy group, the R₂N— amido group, or the RCOO— carboxylate group, with R being a hydrocarbyl radical, preferably alkyl, comprising 1 to 30 carbon atoms, x and y can assume the integer values of 0 to 4, and z is equal to 0 or 0.5, with the sum: x+y+2z being equal to 4. By way of examples, it is possible to cite the zirconium halides such as zirconium tetrachloride ZrCl₄, zirconium tetrabromide ZrBr₄, the alcoholates such as zirconium tetrapropylate Zr(OC₃H₇)₄, zirconium tetrabutylate Zr(OC₄H₉)₄, the carboxylates such as zirconium tetra(2-ethylhexanoate) Zr(OCOC₇H₁₅)₄ or the oxo-carboxylates such as dizirconium oxo-hexaethyl-2-hexanoate [Zr(OCOC₇H₁₅)₃]₂O.

The organic compounds that are selected from among the class of acetals and cetals that are used according to the invention result from the condensation of an aldehyde or a ketone with a monoalcohol or a polyalcohol, for example a glycol. They correspond to the following general formula:

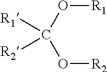

in which R1′ and R2′ consist of a hydrogen atom or a hydrocarbyl radical comprising 1 to 30 carbon atoms, and R1 and R2 are hydrocarbyl radicals comprising 1 to 30 carbon atoms. The two radicals R1′ and R2′ and the two radicals R1 and R2 can be identical or different. They can also be part of a cycle. By way of examples, it is possible to cite diethoxymethane, diisopropoxymethane, diethoxy-1,1-ethane, diisobutoxy-1,1-ethane, dimethoxy-1,1-decane, nonyl-2-dioxolane-1,3, dimethoxy-2,2-propane, dibutoxy-2,2-propane, dioctoxy-2,2-propane, dimethoxy-2,2-octane, dimethoxy-1,1-cyclohexane, di-(ethyl-2-hexyloxy)-2,2-propane.

The aluminum compounds used in the invention are represented by the general formula AlR″ₙX₃₋ₙ, in which R″ is a hydrocarbyl radical, preferably alkyl, comprising 1 to 6 carbon atoms, X is a chlorine or bromine atom, preferably a chlorine atom, and n is a number between 1 and 2, able to be equal in particular to 1, to 1.5, or to 2. By way of examples, it is possible to cite chlorodiethyl aluminum, dichloroethyl aluminum, ethylaluminum sesquichloride, or mixtures thereof.

The components of the catalytic system can be brought into contact in any order within a hydrocarbon, for example a hydrocarbon that is saturated such as hexane or heptane and/or an aromatic hydrocarbon such as toluene, and/or one or more oligomerization by-products such as the higher-order oligomers. Preferably, the zirconium compound is first mixed with acetal or cetal, and then the aluminum compound is added to the whole.

The molar ratio between the acetal or the cetal and the zirconium compound is from approximately 0.1:1 to 5:1 and preferably from approximately 0.5:1 to 2:1. The molar ratio between the aluminum compound and the zirconium compound is from approximately 1:1 to 100:1, preferably from approximately 5:1 to 50:1. The zirconium concentration in the thus prepared catalytic solution is advantageously between 10⁻⁴ and 0.5 mol per liter, and preferably between 2.10⁻³and 0.1 mol per liter. The temperature at which the mixture of the three components is produced is usually between −10 and +150° C., preferably between 0 and +80° C., and, for example, equal to the ambient temperature (15 to 30° C.). This mixing can be carried out under an atmosphere of cover gas or ethylene.

The thus obtained catalytic solution can be used as such or it can be diluted by adding products of the reaction.

According to this second variant, the reaction for oligomerization of ethylene can be implemented at a temperature of 20 to 180° C., preferably 130 and 150° C., under pressure conditions such that the reagents are kept at least for the most part in the liquid phase or in the condensed phase. The pressure is generally between 0.5 and 15 MPa, preferably between 7 MPa and 9 MPa. The contact time is generally between 0.5 and 20 hours, preferably between 1 and 2 hours.

In a particular method for implementing this catalytic reaction variant for intermittent oligomerization, a volume selected from the catalytic solution, prepared as described above, is introduced into a reactor that is equipped with conventional stirring and cooling systems, and then it is pressurized by ethylene at a pressure that is generally between 0.5 and 15 MPa, preferably between 7 and 9 MPa; the temperature in general is maintained between 20 and 180° C., preferably between 130 and 150° C. The oligomerization reactor is supplied by ethylene at constant pressure until the total volume of liquid that is produced represents between 2 and 50 times the volume of the catalytic solution that was originally introduced. The catalyst is then destroyed, for example by adding water, and the products of the reaction and the optional solvent are drawn off and separated.

In the case of continuous operation, the implementation is preferably as follows: the catalytic solution is injected at the same time as ethylene into a reactor that is stirred by conventional mechanical means or by external recirculation. It is also possible to inject the components of the catalyst separately into the reaction medium, for example the production of interaction of the zirconium compound with acetal (or cetal), on the one hand, and hydrocarbyl-aluminum halide, on the other hand. The temperature is maintained between 20 and 180° C., preferably between 130 and 150° C., and the pressure is generally adjusted between 0.5 and 15 MPa, preferably between 7 and 9 MPa. A portion of the reaction mixture flows through a pressure-reducing valve, which keeps the pressure constant, at a mass flow rate that is equal to the mass flow rate of the fluids that are introduced. The thus expanded fluid is sent into a separation system that makes it possible to separate an n-butene-enriched fraction from ethylene and C5+ oligomers (n-hexenes, n-octenes, n-decenes), on the one hand, ethylene that can be returned to the reactor, and then, if necessary, the C5+ oligomers between them, on the other hand. The heavy products that contain the catalyst can be incinerated.

In a preferred method, the oligomerization reaction is implemented in a solvent. In a preferred way, the solvent is selected from among the group of aliphatic and cycloaliphatic hydrocarbons such as hexane, cyclohexane or heptane, or in an aromatic hydrocarbon such as xylenes and preferably ortho-xylene, and toluene.

In the case of this second variant, the oligomerization is carried out with a catalytic system that comprises a catalyst that is based on zirconium and an aluminum compound.

The conversion per pass is then generally between 35 and 40%. The oligomers that are formed comprise between 25 and 35% of n-butenes. The n-butenes for the most part consist of butene-1 (>98%).

The mixture of n-butenes generally comprises less than 0.5% by weight, and even less than 0.2% by weight of isobutene.

According to the first or second variant of the process according to the invention that is implemented, the conversion per pass is therefore generally between 35% and 98%, and the selectivity of n-butenes is generally between 25% and 80%.

The effluent of the oligomerization of the ethylene that is obtained by the two types of catalyst therefore primarily contains n-butenes, and oligomers with 6 carbon atoms and more, as well as unreacted ethylene. The effluent contains very small quantities of isobutene.

b/ Separation of an n-Butene-Enriched Fraction

The effluent that is obtained in stage a) for oligomerization of ethylene is subjected to a separation stage in such a way as to obtain an n-butene-enriched fraction. Thus, the separation stage b) can lead to one or more fractions that are low in n-butenes.

The separation stage can be carried out by evaporation, distillation, extractive distillation, extraction by solvent or else by a combination of these techniques. These processes are known by one skilled in the art. Preferably, a separation of the effluent that is obtained in stage a) is carried out by distillation.

In a preferred manner, the effluent of the oligomerization reactor is sent into a distillation column system comprising one or more columns that makes it possible to separate, on the one hand, n-butenes from ethylene, which can be returned to the oligomerization reactor, and, on the other hand, the other by-products with 6 carbon atoms and more, and even optionally a solvent, such as, for example, a saturated hydrocarbon, an aromatic compound, or a mixture of n-butenes of a composition that is preferably close to or identical to that of the effluent and of which a portion can be returned into the section for preparation of the catalyst.

c/ Oxidizing Dehydrogenation of n-Butenes into 1,3-Butadiene

The third stage of the process according to the invention comprises the dehydrogenation of the n-butene-enriched fraction obtained in stage b) into 1,3-butadiene in the presence of a heterogeneous catalyst.

Preferably, oxidizing dehydrogenation of said n-butene-enriched fraction that is obtained in stage b) is carried out by bringing at least one portion of said effluent into contact with a heterogeneous catalyst in the presence of oxygen and water vapor, in such a way as to produce an effluent that comprises 1,3-butadiene.

The oxidizing dehydrogenation of n-butene is a reaction between n-butene and oxygen that produces 1,3-butadiene and water.

In addition to the n-butene feedstock, the oxidizing dehydrogenation stage requires a supply of oxygen. The source of oxygen can be pure oxygen, air, or oxygen-enriched air.

The oxidizing dehydrogenation reaction is preferably done in the presence of vapor. The vapor makes it possible to activate the catalyst, eliminates the coke from the catalyst by means of a gas reaction with water, or acts to dissipate heat from the reaction.

The oxidizing dehydrogenation of butenes into butadiene can be carried out by any catalytic process that is known from the prior art, and among the latter, those that lead to a high selectivity of butadiene are preferably selected.

The catalysts that are used in the oxidizing dehydrogenation of n-butene, known as of this time, are catalysts based on transition metal oxides, in particular catalysts based on oxides of molybdenum and bismuth, based on ferrite, or else based on oxides of tin and phosphorus. The catalysts may or may not be deposited on a substrate.

According to a first variant, the oxidizing dehydrogenation is implemented in the presence of a catalytic system comprising a catalyst based on oxides of molybdenum and bismuth. The catalysts are particularly well suited for oxidizing dehydrogenation and are generally based on a multi-metal oxide Mo—Bi—O system that also in general comprises iron.

In general, the catalyst also comprises additional components from among groups 1 to 15 of the periodic table, for example potassium, magnesium, zirconium, chromium, nickel, cobalt, cadmium, tin, lead, germanium, lanthanum, manganese, tungsten, phosphorus, cerium, aluminum or silicon.

The composition of a multitude of catalysts of multi-metal oxides that are suitable for oxidizing dehydrogenation can be included in general formula (I)

Mo₁₂BiₐFe_{b}Co_{c}Ni_{d}CrₑX¹_{f}K_{g}Oₓ (I)

in which the variables are each defined as follows: X¹═O, Sn, Mn, La, Ce, Ge, Ti, Zr, Hf, Nb, P, Si, Sb, Al, Cd and/or Mg, a=from 0.5 to 5, preferably from 0.5 to 2; b=from 0 to 5, preferably from 2 to 4; c=from 0 to 10, preferably from 3 to 10; d=0 to 10; e=0 to 10, preferably from 0.1 to 4; f=0 to 5, preferably from 0.1 to 2; g=0 to 2, preferably from 0.01 to 1, and x=a number that is determined by the valence and the frequency of the elements of (I) other than oxygen.

Among the multi-component catalysts based on bismuth molybdate, it is possible to use, for example, catalysts such as mixed oxides composed of Ni, Cs, Bi, Mo (described in the document of M W J. Wolfs, Ph.A. Batist, J. Catal., Vol. 32, p. 25 (1974)), catalysts such as mixed oxides composed of Co, Fe, Bi, Mg, K, Mo (described in U.S. Pat. No. 3,998,867), catalysts of mixed oxides composed of Ni, Co, Fe, Bi, P, K, Mo (described in U.S. Pat. No. 3,764,632); catalysts of mixed oxides composed of four metal elements comprise a metallic divalent cationic component (for example, Ca, Mg, Fe, Mn, Sr, Ni), a metal trivalent cation component (for example, Fe, Al), Bi and Mo (described in WO2008/147055).

Other suitable catalysts are described in the documents US2008/0119680 or U.S. Pat. No. 4,423,281 such as Mo₁₂BiNi₈Pb_{0.5}Cr₃K_{0.2}Oₓ, Mo₁₂BiNi₇Al₃Cr_{0.5}K_{0.5}Oₓ, Mo₁₂BiNi₆Cd₂Cr₃P_{0.5}Oₓ, Mo₁₂BiNi_{0.5}Cr₃P_{0.5}Mg_{7.5}K_{0.1}Oₓ+SiO₂), Mo₁₂BiCo_{4.5}Ni_{2.5}Cr₃P_{0.5}K_{0.1}Oₓ, Mo₁₂BiFe_{0.1}Ni₈ZrCr₃K_{0.2}Oₓ, Mo₁₂BiFe_{0.1}Ni₈AlCr₃K_{0.2}Oₓ, Mo₁₂BiFe₃Co_{4.5}Ni_{2.5}P_{0.5}K_{0.1}Oₓ+SiO₂), (Mo₁₂BiFe₃Co_{4.5}Ni_{2.5}Cr_{0.5}K_{0.1}Oₓ and Mo_{13.75}BiFe₃Co_{4.5}Ni_{2.5}Ge_{0.5}K_{0.8}Oₓ.

It is also possible to use the catalysts that are described in JP2011/148720 using a multi-metal oxide Mo—B—O system that comprises molybdenum, bismuth, iron and cobalt.

According to a second variant, the oxidizing dehydrogenation is implemented in the presence of a catalytic system comprising a ferrite-based catalyst. This type of catalyst found its application in the Oxo-D™ process of Petro-Tex, described in the document of Welch, L. M.; Groce, L. J.; Christmann, H. F., Hydrocarbon Processing, 131, November, 1978.

The ferrite-based catalysts are, for example, MgFe₂O₄, CoFe₂O₄, CuFe₂O₄, MnFe₂O₄, ZnFe₂O₄, ZnCrFe₂O₄, and MgCrFe₂O₄.

According to a third variant, the oxidizing dehydrogenation is implemented in the presence of a catalytic system comprising a catalyst based on oxides of tin and phosphorus. This type of catalyst found its application in the O-X-D™ process of Phillips Petroleum Company (described in U.S. Pat. No. 3,320,329).

The catalysts that are based on oxides of tin and phosphorus (commonly referred to as Sn—P—O catalysts) can comprise additional components. It is possible, for example, to cite the catalysts Mg—Sn—P—O, Ba—Sn—P—O (described in U.S. Pat. No. 3,789,078) or Ca—Sn—P—O, Na—Sn—P—O, K—Sn—P—O, and Rb—Sn—P—O (described in U.S. Pat. No. 3,925,499) and Li—Sn—P—O (described in the document of Hutson, T.; Skinner, R. D.; Logan, R. S., Hydrocarbon Processing, 133, June, 1974).

It is also possible to use combinations of different types of catalysts, as described in, for example, EP2256101.

The oxidizing dehydrogenation reaction can be implemented at a temperature of from 300 to 650° C., preferably from 310 to 550° C., and in a more preferred manner from 320 to 460° C. The pressure is generally 0.01 MPa and 2 MPa, preferably from 0.01 MPa to 0.5 MPa, and in a more preferred manner from 0.05 MPa to 0.3 MPa. The mass flow rate of the feedstock relative to the mass of the bed of the catalyst (PPH or WHSV) is generally from 0.1 to 10 h⁻¹, preferably from 0.2 to 5 h⁻¹.

The oxygen (in its O₂ form)/n-butenes molar ratio is generally between 0.5 and 0.75, preferably between 0.55 and 0.70.

The vapor/n-butene molar ratio is generally from 10 to 20:1.

The conversion of n-butenes is generally from 75 to 85% by weight, and the yield of 1,3-butadiene is generally from 60 to 85%.

Oxidizing dehydrogenation can be performed in any type of reactors such as the fixed-bed, boiling-bed or moving-bed reactors. Preferably, a fixed-bed reactor is used.

The oxidizing dehydrogenation reaction can be carried out in a reaction section that can comprise one or more reactors, with the reactors able to be identical or different.

d/ Separation (Optional Stage)

The effluent from the oxidizing dehydrogenation is a fraction that is highly enriched in butadiene. It can contain traces of unreacted n-butenes. The effluent that comprises 1,3-butadiene that is obtained in stage c) is preferably subjected to at least one separation stage in such a way as to obtain a 1,3-butadiene-enriched fraction.

Preferably, the effluent that comprises 1,3-butadiene can be subjected to a separation by distillation, extractive distillation, extraction by solvent, or else by a combination of these techniques. These processes are known by one skilled in the art. Such separation techniques are described in, for example, “Butadiene, Product Stewardship Guidance Manual,” Revision of Mar. 10, 2002 available on http://www.dow.com/productsafety/pdfs/butadiene_guide.pdf.

The distillation is conceivable to the extent that the conversion per pass of butene-1 is sufficient, taking into account the very close boiling points of butene-1 and 1,3-butadiene.

The main extractive agents used in the extractive distillation are N-methyl-2-pyrrolidone (NMP), dimethylformamide (DMF), acetonitrile, or else an aqueous solution of methoxy-propionitrile (MOPN)/furfural.

The extraction by solvent makes it possible to extract butadiene in the solvent that constitutes the extract. The fraction of butenes is insoluble in the solvent. Solvents such as N-methyl-2-pyrrolidone (NMP), dimethylformamide (DMF) or acetonitrile (ACN) are preferably used.

Butadiene with a purity of greater than 99% is thus obtained.

### BRIEF DESCRIPTION OF DRAWINGS

FIG. 1 illustrates a preferred embodiment of the process according to the invention. Essentially, the installation and the process according to the invention are described.

Ethylene brought to the necessary operating pressure by means of, for example, a compressor and the catalytic system based on a homogeneous catalyst and co-catalyst and, if necessary, a recycling of solvent are introduced by the lines (2) and (4) and (23) respectively in the oligomerization reactor (6). The reactor (6) can be equipped with conventional stifling and cooling systems. The reaction for production of n-butenes and other molecules of longer carbon-containing chains in the liquid phase, as described in stage a), takes place in the reactor (6). In the reactor (6), the temperature is preferably kept between 130 and 150° C. in the case of a zirconium-based catalytic system and preferably between 40 and 60° C. in the case of a nickel-based catalytic system. The pressure is preferably kept at a value that is adequate so that all of the compounds are in the liquid phase (preferably between 7 and 9 MPa in the case of a zirconium-based catalytic system and preferably between 0.5 and 3 MPa in the case of a nickel-based catalytic system). The effluent that is evacuated from the reactor (6) via the line (8) contains the catalytic system, n-butenes, unreacted ethylene, and other oligomers (in particular C6, C8, and C10). The effluent is sent via the line (8) into a system for deactivating and separating catalysts (10). The deactivation of the catalytic system can be carried out according to requirements by injection of ammonia, caustic soda, or an amine. The separation makes it possible to separate, on the one hand, spent catalysts, and, on the other hand, the hydrocarbons that are contained in the effluent (8), for example by operations of evaporation or washing cycles by means of caustic soda and/or water. The spent catalyst is evacuated from the system (10) via the line (12).

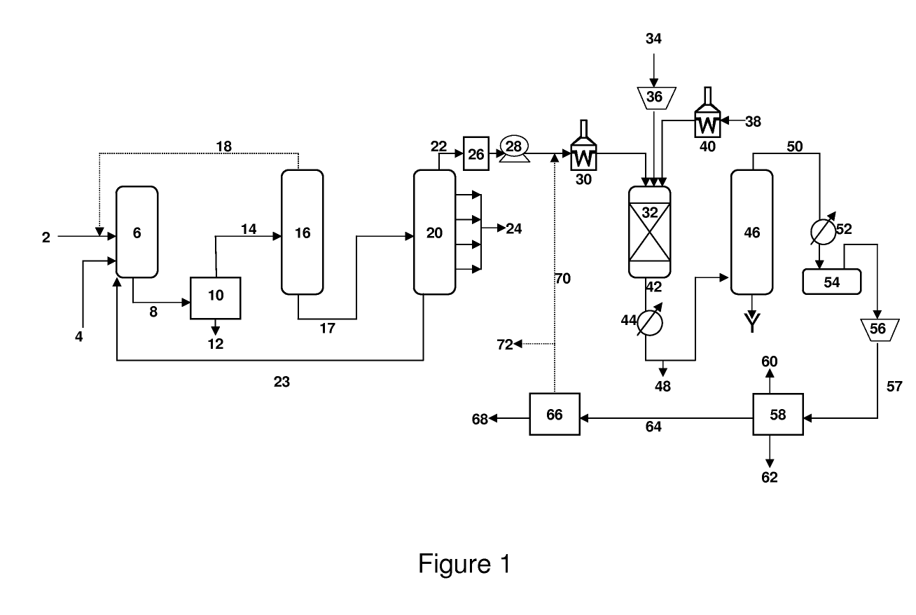

The hydrocarbon-containing fraction that is separated in (10) is sent via the line (14) into a separation section in such a way as to carry out stage b) for recovering the hydrocarbon-containing fraction that contains the n-butenes. For example, the separation section preferably consists of one or more distillation columns that make it possible to evaporate and to fractionate the hydrocarbon-containing fraction. With reference to FIG. 1, two groups of distillation columns (16) and (20) are used. The hydrocarbon-containing fraction is introduced via the line (14) into the first distillation column (16). The column (16) makes it possible to obtain an unconverted ethylene-enriched gaseous fraction that can be recycled, if necessary, via the line (18) to be remixed with the fresh ethylene feedstock that comes in via the pipe (2), to undergo the necessary pressurization, and that is ultimately introduced into the oligomerization reactor (6). The heavy effluent that is obtained at the bottom of the first distillation column (16) and containing the n-butenes and the other oligomers and optionally solvent is sent via the line (17) into a second distillation column (20). A column of the second group of distillation columns (20) makes it possible to recover, at the top of the column, an n-butene-enriched fraction via the line (22). The other oligomers that are formed in the reactor are evacuated in the liquid fraction via the lines (24). They are, if necessary, separated successively into various fractions, C6+C8, solvent and C10+, for example, by means of successive additional fractionation columns. The optional solvent can be recycled via the line (23) in the oligomerization reactor (6). An optional storage chamber (26) makes possible an optional storage of n-butenes (22) that are obtained at the top of the column (20).

To carry out the oxidizing dehydrogenation as described in stage c), the n-butene-enriched fraction, obtained by oligomerization of ethylene and optionally stored in the chamber (26), is pressurized by the pump (28), preheated in the furnace (30) and introduced into the oxidizing dehydrogenation reactor (32). A stream containing oxygen, for example air, pressurized by the compressor (36), is also introduced via the line (34) as well as via the line (38) of the water vapor, preheated in the furnace (40). The oxidizing dehydrogenation reaction is generally implemented at a temperature of from 300 to 650° C., at a pressure of 0.01 MPa and 2 MPa, and with a mass flow rate of the feedstock relative to the mass of the catalyst bed (PPH or WHSV) of 0.1 to 10 h⁻¹. The oxidizing dehydrogenation can be carried out in any type of reactors such as the fixed-bed, boiling-bed or moving-bed reactors. The reactor that is presented in the figure is a fixed-bed reactor. The oxidizing dehydrogenation reaction can be carried out in a reaction section that can comprise one or more reactors, with the reactors able to be identical or different.

The effluent that is evacuated from the oxidizing dehydrogenation reactor via the line (42) contains 1,3-butadiene as well as small quantities of unreacted n-butenes, polymers, and light gases (hydrogen and light hydrocarbons C2, C3). This effluent can be cooled and compressed.

For example, with reference to FIG. 1, the effluent that circulates in the line (42) is cooled in a heat exchanger system (44) and then sent into a quenching system (46). The effluent that is obtained from the quenching (50) is cooled in a heat exchanger system (52), collected in the chamber (54), and compressed in the compressor (56) to obtain a cooled and compressed effluent that circulates in the pipe (57).

The effluent (57) that contains 1,3-butadiene can optionally undergo a separation stage as described in stage d). The separation can be carried out by distillation, extractive distillation, extraction by solvent, or else by a combination of these techniques.

For example, with reference to FIG. 1, the effluent is introduced via the pipe (57) into a separation system (58) for separating the light gases that are evacuated via the line (60), the polymers that are formed via the line (62), and a 1,3-butadiene-enriched stream in liquid form evacuated via the line (64).

The 1,3-butadiene-enriched stream that contains traces of unreacted n-butenes and that is obtained via the line (64) can also be subjected to a separation stage in the chamber (66), for example by extraction with solvents, making it possible to recover high-purity 1,3-butadiene via the line (68). At least a portion of n-butenes recovered during the separation stage in the chamber (66) can advantageously be recycled via the line (70) in the oxidizing dehydrogenation stage for supplying the reactor (32). Alternately, at least a portion of the n-butenes can be drawn off for any other use via the line (72).

The entire disclosures of all applications, patents, and publications, cited herein and of corresponding application number FR 12/02.510, filed Sep. 21, 2012 are incorporated by reference herein.

The following examples illustrate the invention without limiting its scope:

### EXAMPLE 1

### Oligomerization with a Homogeneous Catalytic System Based on a Nickel Compound and an Aluminum Compound

The feedstock that is used in this example for the most part comprises ethylene that is obtained from a steam-cracking device. Its composition is as follows:

| | | |
|---|---|---|
| Ethylene | % by Volume | 99.95 |
| Saturated Paraffins (Including Methane, Ethane) | % by Volume | 0.05 |

The feedstock is subjected to an oligomerization by a catalyst based on nickel and an aluminum compound. The effluent of the oligomerization unit is separated by distillation for recovering an n-butene-rich fraction: this n-butene-rich fraction is then sent into an oxidizing dehydrogenation unit with water and air.

In the homogeneous catalysis stage, the catalytic system comprises a catalyst based on an Ni 2-ethylhexanoate complex with 13% by weight of Ni, trifluoroacetic acid, and a co-catalyst activator that comprises dichloroethyl aluminum. The molar ratio between trifluoroacetic acid and Ni is equal to 1.02:1, and the Al/Ni molar ratio is 15:1. A dwell time of 4 hours is used. The reactor is operated at 50° C. and 2.5 MPa.

In the dehydrogenation stage, the catalytic system comprises a catalyst based on bismuth and molybdenum (Co₉Fe₃Bi₁Mo₁₂O₅₁) prepared according to the process that is described in the patent application EP-A-2 256 101, performed with a PPH of 2 hr⁻¹, expressed relative to the C4 liquid feedstock, a pressure of 0.15 MPa, and a temperature of 430° C.

| Mass | Homogeneous Catalysis | | Dehydrogenation Stage | |
|---|---|---|---|---|
| Balance | Stage (Stage a) | | (Stage c) | |
| Ethylene | 100 | | | |
| Co-Catalysts + | 0.1 | | | |
| Deactivation | | | | |
| Agent | | | | |
| n-Butenes | | 57 | 57 | |
| Light Purges | | 5 | | |
| Other | | 38 | | |
| Oligomers | | | | |
| with More | | | | |
| than 5 | | | | |
| Carbon | | | | |
| Atoms | | | | |
| Spent and | | 0.1 | | |
| Heavy | | | | |
| Catalysts | | | | |
| Butadiene | | | | 39.9 |
| Air (1) | | | 87 | |
| Water Vapor (2) | | | 190 | |
| Others | | | | 294.1 |
| Sum | 100.10 | 100.10 | 334 | 334 |

(1): Oxygen (in its O₂ form)/n-butenes molar ratio = 0.60
(2): Vapor/n-butenes molar ratio = 10

The mass yield of n-butenes of the first stage is therefore 57%, and the yield of the second stage is 70%. The overall mass yield of butadiene from ethylene is 39.9%.

### EXAMPLE 2

### Oligomerization with a Homogeneous Catalytic System Based on a Zirconium Compound and an Aluminum Compound

The feedstock that is used is that of Example 1.

The feedstock is subjected to an oligomerization in the presence of a catalyst based on zirconium and an aluminum compound. The effluent of the oligomerization unit is separated by distillation for recovering an n-butene-rich fraction; this n-butene-rich fraction is then sent into a unit for oxidizing dehydrogenation with water and air.

In the homogeneous catalysis stage, the catalytic system comprises zirconium chloride (ZrCl₄), di-(ethyl-2-hexyloxy)-2,2-propane, and a co-catalyst activator that comprises ethylaluminum sesquichloride, which are introduced with mass ratios respectively of 1:1 and 2:10. A dwell time of 1.5 hours is used. The reactor is operated at 140° C. and 8.0 MPa.

In the dehydrogenation stage, the catalytic system comprises a catalyst based on bismuth and molybdenum (Co₉Fe₃Bi₁Mo₁₂O₅₁) that is prepared according to the process described in the patent application EP-A-2 256 101, performed with a PPH of 2 h⁻¹, expressed relative to the C4 liquid feedstock, a pressure of 0.15 MPa, and a temperature of 430° C.

| Mass | Homogeneous Catalysis | | Dehydrogenation Stage | |
|---|---|---|---|---|
| Balance | Stage (Stage a) | | (Stage c) | |
| Ethylene | 100.00 | | | |
| Co-Catalysts + | 1.04 | | | |
| Deactivation | | | | |
| Agent | | | | |
| n-Butenes | | 33.5 | 33.5 | |
| Light Purges | | 2.50 | | |
| Other | | 62.2 | | |
| Oligomers | | | | |
| with More | | | | |
| than 5 | | | | |
| Carbon | | | | |
| Atoms | | | | |
| Spent and | | 2.9 | | |
| Heavy | | | | |
| Catalysts | | | | |
| Butadiene | | | | 23.5 |
| Air (1) | | | 51.1 | 172.9 |
| Water Vapor (2) | | | 111.7 | |
| Others | | | 0 | |
| Sum | 101.04 | 101.04 | 196.4 | 196.4 |

(1): Oxygen (in its O₂ form)/n-butenes molar ratio = 0.60
(2): Vapor/n-butenes molar ratio = 10

The mass yield of n-butenes of the first stage is therefore 33.5%, and the yield of the second stage is 70%. The overall mass yield of butadiene from ethylene is 23.5%.

1. Process for the production of 1,3-butadiene from a stream that comprises ethylene implementing the following stages:
a) An oligomerization of ethylene into oligomers is carried out by bringing said stream into contact with a catalytic system based on a homogeneous catalyst, in such a way as to produce an effluent that comprises n-butenes and oligomers with 6 carbon atoms and more including n-hexenes, n-octenes, and n-decenes,
b) A separation of the effluent obtained in stage a) is carried out in such a way as to obtain an n-butene-enriched fraction,
c) Dehydrogenation of said n-butene-enriched fraction obtained in stage b) is carried out by bringing at least a portion of said effluent into contact with a heterogeneous catalyst, in such a way as to produce an effluent comprising 1,3-butadiene.

2. Process according to claim 1, in which the oligomerization of ethylene is implemented in the presence of a catalytic system that comprises:
i) At least one bivalent nickel compound,
ii) At least one hydrocarbyl aluminum dihalide of formula AlRX₂, in which R is a hydrocarbyl radical comprising 1 to 12 carbon atoms, such as alkyl, aryl, aralkyl, alkaryl or cycloalkyl, X is a chlorine or bromine atom, and
iii) Optionally at least one Brønsted organic acid.

3. Process according to claim 2, in which said bivalent nickel compound is a nickel carboxylate of general formula (R₁COO)₂Ni, where R₁ is an alkyl, cycloalkyl, alkenyl, aryl, aralkyl or alkaryl radical, containing up to 20 carbon atoms.

4. Process according to claim 2, in which said Brønsted organic acid has a pKa at 20° C. that is at most equal to 3 and is selected from the group that is formed by the halocarboxylic acids of formula R₂COOH, in which R₂ is a halogenated alkyl radical containing at least one alpha-halogen atom of the group —COOH with 2 to 10 carbon atoms in all.

5. Process according to claim 2, in which the hydrocarbyl aluminum dihalide is enriched with an aluminum trihalide, with the mixture of the two compounds corresponding to the formula AlRₙX₃₋ₙ, in which R is a hydrocarbyl radical comprising 1 to 12 carbon atoms, such as alkyl, aryl, aralkyl, alkaryl or cycloalkyl, X is a chlorine or bromine atom, and n is a number between 0 and 1.

6. Process according to claim 2, in which the oligomerization of ethylene is implemented at a temperature of −20 to 80° C., at a pressure of between 0.5 MPa and 5 MPa, and with a contact time of between 0.5 and 20 hours.

7. Process according to claim 1, in which the oligomerization of ethylene is implemented in the presence of a catalytic system comprising:
i) At least one zirconium compound of formula ZrXₓY_{y}O_{z} in which X is a chlorine or bromine atom, Y is a radical that is selected from the group that is formed by the RO— alkoxys, the R₂N— amidos, and the RCOO— carboxylates, where R is a hydrocarbyl radical comprising 1 to 30 carbon atoms, and x and y can assume the integer values of 0 to 4, and z is equal to 0 or 0.5, with the sum x+y+2z being equal to 4,
ii) At least one organic compound of formula 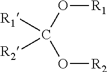
in which R′₁ and R′₂ consist of a hydrogen atom or a hydrocarbyl radical comprising 1 to 30 carbon atoms; R₁ and R₂ are hydrocarbyl radicals comprising 1 to 30 carbon atoms,
iii) And at least one aluminum compound of formula AlR″ₙX₃₋ₙ, in which R″ is a hydrocarbyl radical comprising 1 to 6 carbon atoms, X is a chlorine or bromine atom, and n is a number between 1 and 2.

8. Process according to claim 7, in which the oligomerization of ethylene is implemented at a temperature of 20 to 180° C., at a pressure of between 0.5 MPa and 15 MPa, and with a contact time of between 0.5 and 20 hours.

9. Process according to claim 1, in which in stage b), a separation of the effluent that is obtained in stage a) is carried out by distillation.

10. Process according to claim 1, in which, in stage c), an oxidizing dehydrogenation of said n-butene-enriched fraction that is obtained in stage b) is carried out by bringing at least a portion of said effluent into contact with a heterogeneous catalyst in the presence of oxygen and water vapor, in such a way as to produce an effluent comprising 1,3-butadiene.

11. Process according to claim 10, in which the oxidizing dehydrogenation is implemented in the presence of a catalytic system that comprises a catalyst based on oxides of molybdenum and bismuth.

12. Process according to claim 10, in which the oxidizing dehydrogenation is implemented in the presence of a catalytic system comprising a ferrite-based catalyst.

13. Process according to claim 10, in which the oxidizing dehydrogenation is implemented in the presence of a catalytic system that comprises a catalyst based on oxides of tin and phosphorus.

14. Process according to claim 10, in which the oxidizing dehydrogenation is implemented at a temperature of 300 to 650° C., at a pressure of 0.01 MPa to 2 MPa, and at a mass flow rate of the feedstock relative to the mass of the bed of the catalyst of 0.1 to 10 h⁻¹.

15. Process according to claim 1, in which the effluent that comprises the 1,3-butadiene obtained in stage c) is subjected to at least one separation stage in such a way as to obtain a 1,3-butadiene-enriched fraction.
